(19) United States
(12) Patent Application Publication
(10) Pub. No.: US 2005/0164331 A1
(43) Pub. Date: Jul. 28, 2005

(54) Method and device for detecting toxic material in water using microbial fuel cell

(75) Inventors: Hyung Joo Kim, Seoul (KR); Dae Won Choi, Seoul (KR); Moon Sik Hyun, Seoul (KR); Sung Hyun Nam, Seoul (KR)

Correspondence Address: SQUIRE, SANDERS & DEMPSEY L.L.P, 600 HANSEN WAY, PALO ALTO, CA, 94304-1043, US

(73) Assignee: Korea Biosystems Corp., Seoul (KR)

(21) Appl. No.: 10/509,718

(22) PCT Filed: Apr. 26, 2003
(86) PCT No.: PCT/KR03/00854

(30) Foreign Application Priority Data
Apr. 27, 2002 (KR) 10-2002-0023232

(51) Int. Cl. C12Q 1/02; C12M 1/34

(52) U.S. Cl. 435/29; 435/287.1

(57) ABSTRACT

The present invention relates to a method and device for detecting the toxic materials in water by using an electrochemically active microorganism. Described in details, the present invention comprises the steps of: determining the electrical signals, generated by the microbial fuel cell; introducing the sample into the above-mentioned fuel cell; and determining the degree of changes in the electrochemical signals, generated by the above-mentioned microbial fuel cell, in order to provide a method for detecting the toxic materials in water. Thus, according to the present invention, when toxic materials are present in the sample for determination, generation of electricity by the electrically active bacteria in the microbial fuel cell is decreased remarkably, maximizing the sensitivity in determining the toxic materials. The use of the microbial fuel cell minimizes the cost and the personnel for the management and the maintenance of the sensor part.

### TECHNICAL FIELD

This invention relates to the detecting method of the toxic materials by a biological means and the device thereof. More specifically, this invention relates to an automatic detecting method of the toxic materials using microbial fuel cell and an automatic alarming device thereof.

### BACKGROUND ART

Until now, the early detection and alarming device for the entry of the toxic materials in water has been developed by many researchers. There are chemical and biological detection devices in the conventional devices for determining toxic materials in water. The chemical detection device has limitation because of many materials present in water and only a few materials are quantitatively determined. It has the disadvantage of necessitating costly instruments and highly skilled engineers for the detection. In order to implement this limitation, various biological devices for detecting toxic materials present in water, have been developed.

Representative conventional biological detecting devices for toxic materials in water include monitoring methods by using fish, water flea and fluorescent microorganism. The device using fish for water quality monitoring takes advantage of the character of the fish to swim against the flow of water, namely it uses the countercurrent phenomenon for detection. After an anti-escape net is installed and when toxic materials are introduced through the inlet, the fish is effected and its swimming activity slows down. The effected fish is pushed back due to the current, yet the fish, by instinct, will move tail fin vigorously in order to move forward again, the tail fin touching the sensor during the process. This action is transformed into electric signals and recorded. The value of this electric signals are detected by the water quality monitoring device and is used to give alarm or in controlling the flow rate of water by the connected controller. This information is input/output through the monitor or the keyboard. The fish used for the purpose is usually a golden dace belonging to the dace genera in the carp family. The shortfalls of using the fish to detect the toxic material is that the object of detecting the toxicity is so large that it requires 8 hours to determine the toxicity when 8 ppm phenol is introduced to water. The method of using fish for biological toxicity alarm system has low sensibility, and the detection time and the error range are wide. The selection and the growing condition of fish reduces reproducibility and uniformity of the alarming system. The device using water flea for the toxic material senses the activity of the water fleas by infrared sensor. It is based on the swimming activity of the fleas; 20 water fleas are placed into a glass or acrylic test chamber where water to be tested is introduced and discharged. The fleas react when water is introduced. While water without toxic materials is in, they show regular activity, yet if toxic materials are contained, their movement becomes irregular and vigorous. The more vigorous activity they show, the more frequently they hit the infrared sensor, making the electrical signal value increase. The temperature sensor determines the temperature all the time and the infrared sensor is controlled by the electronic controller, displaying the value through an output device. The detected water is discharged through water outlet. The early alarming device by water fleas is more sensitive than the device using the fish because the used object is smaller, but maintenance is difficult. When changing the fleas, the test chamber and the various tubes for the input/output of water are either washed or exchanged. Care and efforts are needed in growing the fleas, because the growing water should be prepared and exchanged for 2-3 times a week, and progeny and parent fleas should be carefully separated. The fleas are reared in a special growing chamber where its space is disinfected and any equipments interfering with the growth are eliminated. Fresh air must be supplied to the growing chamber.

The device for automatic detection of the toxicity in water, using the fixed fluorescent microorganism, determines the fluorescence against the toxicity. It needs various light detecting equipments, making it costly and requiring personnel for their maintenance and an expert for them as well.

The present invention is proposed based on the fact that the problems for such conventional automatic detection devices for water toxicity arise eventually from the sensor parts.

### DISCLOSURE OF THE INVENTION

The objective of the present invention is to solve the technical problems in the conventional automatic determination devices for detecting toxicity in water and to provide fast and correct determination method of toxicity with low cost and easy maintenance.

The objective of the present invention is accomplished by the method for detecting the toxic materials in a sample using electrochemically active microorganism.

More specifically, the objective is achieved by the method for detecting the toxic materials in water, characterized in that it comprises the steps of:
determining the electrochemical signals generated from the microbial fuel cell;
introducing a sample into the above-mentioned microbial fuel cell; and
determining the degree of changes in the electrochemical signals generated from the above-mentioned microbial fuel cell. Additionally, the method for detecting toxic materials in water can comprise further a step of screening out the suspension and unwanted materials in the sample before introducing the sample to the above microbial fuel cell.

And the device for detecting the above-mentioned toxic materials comprises a pump taking a sample; a pretreatment tank treating the sample; a microbial fuel cell sensing the changes in the current by the introduced toxic materials; and a PC and a controlling part which control the signal values and automatically determine the toxicity.

In the following, the present invention is illustrated in reference to the drawings. The present invention should be only understood within the scope of the claims and is not limited to the constitutions of the drawings in which:

FIG. 1 is a schematic illustration of the automatic determination device for toxic materials comprising a pump(1) taking a sample; a pretreatment tank(2) treating the sample; a microbial fuel cell(6) sensing the changes in the current occurred by the introduced toxic materials; and a PC and a controlling part(11) which control the value of the signals and automatically determine the toxicity. Additionally, the device comprises a solenoid valve(5) which makes changes of flow of the sample when the toxic materials are introduced and sample-gathering vessel(4) which gathers and stores the sample at a point when the signal is recognized.

The following illustration is the working mechanism of the device with the abovementioned constitution for determining toxic material by the use of the microbial fuel cell. The sample enters into the anode part after passing through the first and the second pretreatment tanks(2,3). The anode part is composed of a carbon felt and platinum(Pt) wire, while its inside part is filled usually with a microorganism catalyst which generates electrochemical energy using organic raw material. The cathode part is filled with ordinary water. Namely, the sample containing organic materials enters into the anode and the water-saturated with air enters the cathode. Now the organic materials are decomposed by microorganism at the anode part of the fuel cell and a current is generated. The current moves along the Pt wire and is measured by the voltmeter. In usual situation, the electric current does not show any range of changes, however, once the toxic material enters into the anode part, metabolism of the electrochemically active microorganism is slowed down, making the abrupt drop in the voltage. Such sharp drop of the current is processed by the PC and the controlling part, making the alarm activated in the audio/video display.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram of the device for detecting the toxic materials.

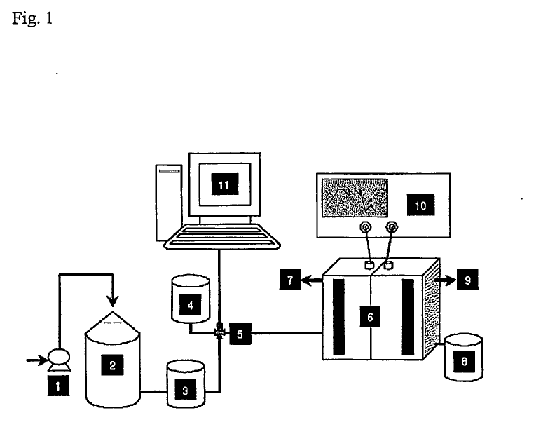

FIG. 2 is the graph showing the result of Embodiment 1.

FIG. 3 is the graph showing the result of Embodiment 2.

FIG. 4 is the graph showing the result of Embodiment 4.

FIG. 5 is the graph showing the result of Embodiment 5.

### DESCRIPTION OF PARTS LIST

| | |
|---|---|
| (1) Sample inlet pump | (2) First pretreatment tank for sample |
| (3) Second pretreatment | (4) Sample-gathering vessel |
| tank for sample | |
| (5) Solenoid valve | (6) Microbial fuel cell |
| (7) Sample outlet | (8) Tap water chamber |
| (9) Tap water exit | (10) Voltmeter |
| (11) PC, controlling part | |

### BEST MODE FOR CARRYING OUT INVENTION

### First Preferred Embodiment

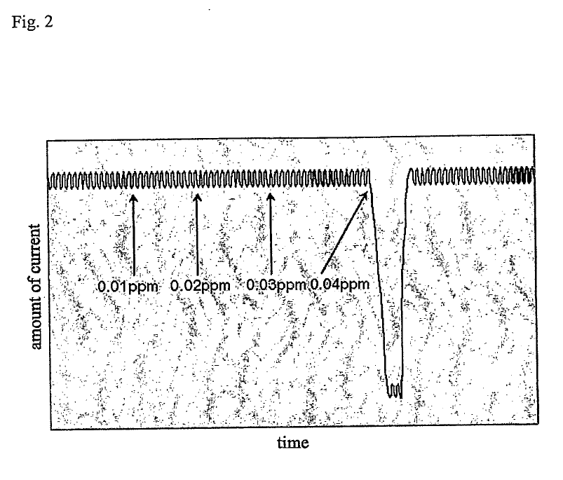

Active sludge was introduced into the anode part so that the electrochemically active bacteria in the sludge are attached to the electrode and densely cultured. To the cathode part, water saturated with air was incorporated, keeping a certain potential difference so as to make an efficient biological electrochemical reaction occur in the microbial fuel cell. Into this microbial fuel cell, added were glucose and glutamic acid(CODcr 200 ppm, which means the chemical oxygen demand due to the potassium dichromate) as fuel and the generated current was measured by the volt meter(2000 multimeter, Keithley Instrument. Inc, USA) at 60 seconds interval, while adding to the used fuel cell, Cr⁶⁺ standard solution successively in the concentration of 0.01 ppm, 0.02 ppm, 0.03 ppm, 0.04 ppm and 0.05 ppm. The result showed a sharp drop of electric current value which was usually generated in constant rate, at 0.04 ppm of Cr⁶⁺ (FIG. 2)

### Second Preferred Embodiment

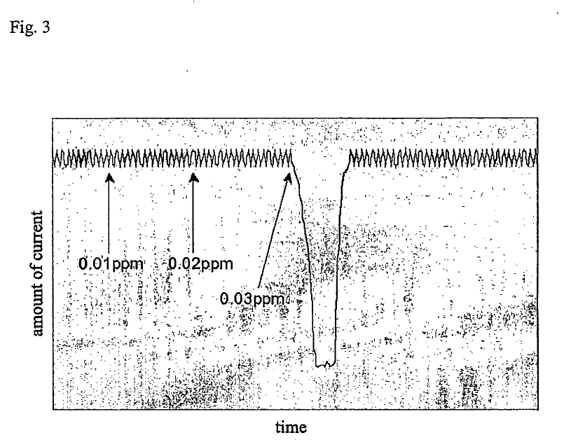

In the Embodiment 2, the same fuel cell and the fuel as in the Embodiment 1 was used. After feeding the fuel, the generated amount of current was measured by the volt meter(2000 multimeter, Keithley Instrument. Inc, USA) at 60 seconds interval, while adding to the used fuel cell, mercury(Hg) standard solution successively in the concentration of 0.01 ppm, 0.02 ppm, 0.03 ppm, 0.04 ppm and 0.05 ppm. The result showed as in Embodiment 1 that the current generated in a constant rate showed a sharp drop at 0.03 ppm of Hg. (FIG. 3)

### Third Preferred Embodiment

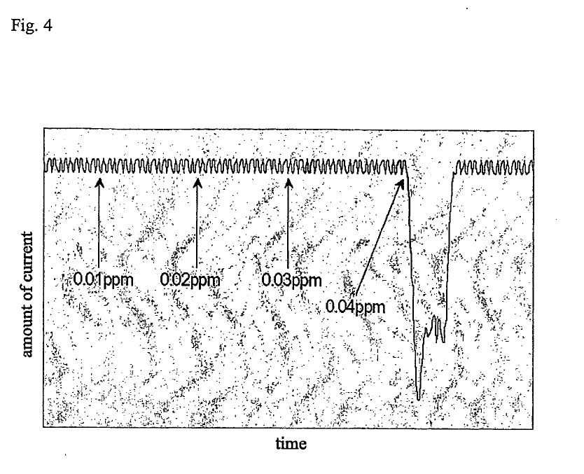

In the Embodiment 3, the same fuel cell and the fuel as in the Embodiment 1 was used. After feeding the fuel, the generated amount of current was measured by the volt meter(2000 multimeter, Keithley Instrument. Inc, USA) at 60 seconds interval, while adding to the used fuel cell, lead(Pb) standard solution successively in the concentration of 0.01 ppm, 0.02 ppm, 0.03 ppm, 0.04 ppm and 0.05 ppm. The result showed as in Embodiment 1 that the current generated in a constant rate showed a sharp drop at 0.04 ppm of Pb. (FIG. 4)

### Fourth Preferred Embodiment

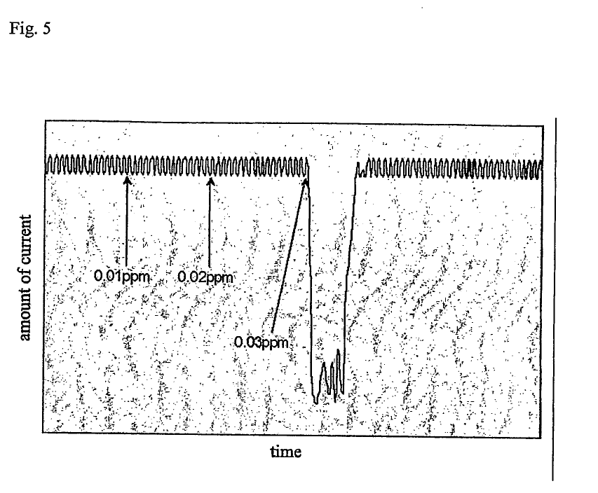

In the Embodiment 4, the same fuel cell and the fuel as in the Embodiment 1 was used. After feeding the fuel, the generated amount of current was measured by the volt meter(2000 multimeter, Keithley Instrument. Inc, USA) at 60 seconds interval, while adding to the used fuel cell, phenol standard solution successively in the concentration of 0.01 ppm, 0.02 ppm, 0.03 ppm, 0.04 ppm and 0.05 ppm. The result showed as in Embodiment 1 that the current generated in a constant rate showed a sharp drop at 0.03 ppm of phenol. (FIG. 5)

### INDUSTRIAL APPLICABILITY

Thus according to the present invention, when the toxic materials are incorporated into the sample to be tested, there is an abrupt drop of the generated electricity by the electrochemically active bacteria in the microbial fuel cell, maximizing the sensitivity in detecting the toxic materials. Use of the microbial fuel cell minimizes the cost and the personnel in managing and maintaining the sensor part as well as remarkably enhancing the reproducibility and the degree of accuracy in detection of the toxic materials compared with the conventional alarming device. Once the entry signal of the toxic materials is sensed by the detecting device, the sample containing the toxic material is taken on the spot and kept in a sealed vessel. The sample is analyzed later quantitatively and qualitatively for tracing the cause of the entry and providing the data to forecast the consequent damage.

The present invention minimizes the damage by detecting the inflow of the toxic material in the early stage. Development of such excellent devices of detecting toxic materials contributes effectively to the national economy in that the related devices can be exported, replacing importation, once they are locally produced.

According to the present invention, the detecting device of the toxic materials using microbial fuel cell controls the degree of biological toxicity of the waste and sewage water and is used to detect rapidly the pollution of the intake water source of the drinking water. When the device is installed in the protected region of the water source, it can effectively prevent in advance the illegal disposal of the polluted materials by the industries and industrial complex facilities.

1. The method for detecting toxic materials in water by using the electrochemically active microorganism.

2. The method for detecting toxic materials in water comprising the steps of:
a. determining the electrochemical signals generated from the microbial fuel cell;
b. introducing a sample to the above microbial fuel cell; and
c. determining the degree of electrochemical signal changes from the microbial fuel cell.

3. The device for detecting toxic materials in water of claim 2 further comprising a step of screening out the suspension and unwanted materials in the sample before introducing the sample to the above microbial fuel cell.

4. The device for detecting toxic materials in water comprising:
a. a sample inlet pump(1);
b. a first pretreatment tank(2) treating the sample;
c. a microbial fuel cell(6) which senses the changes in the current due to the entry of the toxic materials; and
d. a PC and controlling part(11) which control the value of the signals and automatically determine the toxicity.

5. The device for detecting toxic materials in water of claim 4 further comprising a solenoid valve(5) which changes the flow of the sample when sensing the entry of the toxic materials, and sample-gathering vessel(4) which intakes and stores the sample when the entry of toxic materials are sensed.
